(19) United States
(12) Patent Application Publication
(10) Pub. No.: US 2022/0375615 A1
(43) Pub. Date: Nov. 24, 2022

(54) METHOD AND SYSTEM FOR PREDICTING HEALTH RISK

(71) Applicant: SB Solutions Inc., Ulju-gun (KR)

(72) Inventor: Namhwan Sung, Ulju-gun (KR)

(73) Assignee: SB Solutions Inc., Ulju-gun (KR)

(21) Appl. No.: 17/399,901

(22) Filed: Aug. 11, 2021

(30) Foreign Application Priority Data
May 18, 2021 (KR) 10-2021-0063884

(51) Int. Cl. G16H 50/30; G08B 21/18; G16H 50/20; A61B 5/00

(52) U.S. Cl. CPC G16H 50/30; G08B 21/182; G16H 50/20; A61B 5/7278; A61B 5/746

(57) ABSTRACT

Disclosed are a method and system for predicting a health risk. In an embodiment, a method of predicting a health risk may include collecting a health condition index, generating time-series data by accumulating the health condition index at given time intervals, calculating a health condition index prediction value in a future time by inputting the generated time-series data to a health condition index prediction model, comparing the calculated health condition index prediction value with a preset threshold, and generating a danger alert signal when the calculated health condition index prediction value is out of the threshold.

### CROSS-REFERENCES TO RELATED APPLICATIONS

This application is based on and claims priority under 35 U.S.C. 119 to Korean Patent Application No. 10-2021-0063884, filed on May 18, 2021, in the Korean intellectual property office, the disclosures of which are herein incorporated by reference in their entireties.

### TECHNICAL FIELD

The following description relates to a method and system for predicting a health risk.

### BACKGROUND OF THE INVENTION

Examples in which adult-onset diseases, such as diabetes, hyperlipidemia and thrombosis, are increased continue to increase. Such diseases need to be periodically measured using various bio sensors because it is important to continuously monitor and manage the diseases. A common type of bio sensor is a method of injecting, into a test strip, blood drawn from a finger and then quantizing an output signal by using an electrochemical method or a photometry method.

However, about half of diabetes patients have experienced hypoglycemia for the last six months. It was found that 1/3 of the half of the diabetes patients has repeatedly experienced hypoglycemia three times or more. If a diabetic patient does not take sugar within a short time when a hypoglycemia symptom appears, the diabetic patient may go into a hypoglycemia shock, and may lose his or her consciousness or lead to death in severe cases. For this reason, diabetes patients suffer from a fear of a hypoglycemia shock and feel inconvenient to frequently check blood glucose.

### PRIOR ART DOCUMENT NUMBER

Korean Patent No. 10-2185556

### SUMMARY

This summary is provided to introduce a selection of concepts in a simplified form that are further described below in the Detailed Description. This summary is not intended to identify key characteristics of the claimed subject matter, nor is it intended to be used as an aid in determining the scope of the claimed subject matter.

The present disclosure provides a method and system for predicting a health risk, which can predict a change in a future health state and previously give warning when a danger, such as a hypoglycemia shock, dysarteriotony, reduced oxygen saturation, a sudden change in the heart rate, or an abnormal body temperature, is expected.

In an aspect, there is provided a method of predicting, by a computer device including at least one processor, a health risk, including collecting, by the at least one processor, a health condition index, generating, by the at least one processor, time-series data by accumulating the health condition index at given time intervals, calculating, by the at least one processor, a health condition index prediction value in a future time by inputting the generated time-series data to a health condition index prediction model, comparing, by the at least one processor, the calculated health condition index prediction value with a preset threshold, and generating, by the at least one processor, a danger alert signal when the calculated health condition index prediction value is out of the threshold.

According to an aspect, collecting the health condition index may include receiving the health condition index of an object from an external device or measuring the health condition index of the object through a bio sensor.

According to another aspect, generating the time-series data may include generating the time-series data by accumulating the health condition index at given time intervals in a form a two-dimensional array for each type.

According to yet another aspect, the health condition index prediction model may be trained to receive the time-series data obtained by accumulating the health condition index over time and to output a prediction value for a health condition index in at least one future time after the time-series data.

According to yet another aspect, comparing the calculated health condition index prediction value with the preset threshold may include determining that the calculated health condition index prediction value is out of the preset threshold, when the calculated health condition index prediction value is smaller than a preset lower threshold, the calculated health condition index prediction value is greater than a preset upper threshold, or the calculated health condition index prediction value is included in a preset threshold range.

According to yet another aspect, the method of predicting a health risk may further include outputting, by the at least one processor, the generated danger alert signal.

According to yet another aspect, the method of predicting a health risk may further include displaying, by the at least one processor, at least one of the collected health condition index, the calculated health condition index prediction value and the danger alert signal.

According to yet another aspect, the method of predicting a health risk may further include transmitting, by the at least one processor, at least one of the collected health condition index, the calculated health condition index prediction value and the danger alert signal to an external device.

According to yet another aspect, the method of predicting a health risk may further include generating, by the at least one processor, a lifestyle guide by inputting the generated time-series data to a lifestyle guide model.

According to yet another aspect, generating the lifestyle guide may include inputting the generated time-series data to the health condition index prediction model, inputting an output of the health condition index prediction model to the lifestyle guide model again, and generating an output value of the lifestyle guide model as the lifestyle guide.

In an aspect, there is provided a computer device including at least one processor implemented to execute a computer-readable instruction. The at least one processor is implemented to collect a health condition index, generate time-series data by accumulating the health condition index at given time intervals, calculate a health condition index prediction value in a future time by inputting the generated time-series data to a health condition index prediction model, compare the calculated health condition index prediction value with a preset threshold, and generate a danger alert signal when the calculated health condition index prediction value is out of the threshold.

When a risk, such as a hypoglycemia shock, dysarteriotony, reduced oxygen saturation, a sudden change in the heart rate, or an abnormal body temperature, is predicted based on a change in a future health state, a user is previously warned of the risk so that the user can avoid the risk by securing the time to handle the risk.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a diagram illustrating an example of a network environment according to an embodiment of the present disclosure.

FIG. 2 is a block diagram illustrating an example of a computer device according to an embodiment of the present disclosure.

FIG. 3 is a diagram illustrating an example of a system for predicting a health risk according to an embodiment of the present disclosure.

FIG. 4 is a diagram illustrating an example of time-series data according to an embodiment of the present disclosure.

FIG. 5 is a diagram illustrating an example in which HCIs are predicted according to an embodiment of the present disclosure.

FIG. 6 is a concept view in which HCIs are expected through an HCI prediction model according to an embodiment of the present disclosure.

FIG. 7 is a diagram illustrating an example of internal components of a monitoring device according to an embodiment of the present disclosure.

FIG. 8 is a diagram illustrating an example of internal components of a display device according to an embodiment of the present disclosure.

FIG. 9 is a flowchart illustrating an example of a method of predicting a health risk according to an embodiment of the present disclosure.

FIG. 10 is a diagram illustrating an example of lifestyle guides according to an embodiment of the present disclosure.

FIG. 11 is a diagram illustrating an example of a lifestyle guide model according to an embodiment of the present disclosure.

FIG. 12 is a diagram illustrating another example of a lifestyle guide model according to an embodiment of the present disclosure.

FIG. 13 is a diagram illustrating another example of internal components of a monitoring device according to an embodiment of the present disclosure.

FIG. 14 is a diagram illustrating another example of internal components of a display device according to an embodiment of the present disclosure.

### DETAILED DESCRIPTION

While illustrative embodiments have been illustrated and described, it will be appreciated that various changes can be made therein without departing from the spirit and scope of the invention.

Hereinafter, embodiments are described in detail with reference to the accompanying drawings. However, the embodiments may be changed in various ways, and the scope of right of this patent application is not limited or restricted by such embodiments. It is to be understood that all changes, equivalents and substitutions of the embodiments are included in the scope of right.

Terms used in embodiments are merely used for a description purpose and should not be interpreted as intending to restrict the present disclosure. An expression of the singular number includes an expression of the plural number unless clearly defined otherwise in the context. In this specification, it should be understood that a term, such as “include” or “have”, is intended to designate the presence of a characteristic, a number, a step, an operation, a component, a part or a combination of them described in the specification, and does not exclude the existence or possible addition of one or more other characteristics, numbers, steps, operations, components, parts, or combinations of them in advance.

All terms used herein, including technical or scientific terms, have the same meanings as those commonly understood by a person having ordinary knowledge in the art to which an embodiment pertains, unless defined otherwise in the specification. Terms, such as those commonly used and defined in dictionaries, should be construed as having the same meanings as those in the context of a related technology, and are not construed as being ideal or excessive unless explicitly defined otherwise in the specification.

Furthermore, in describing the present disclosure with reference to the accompanying drawings, the same component is assigned the same reference numeral regardless of its reference numeral, and a redundant description thereof is omitted. In describing an embodiment, a detailed description of a related known art will be omitted if it is deemed to make the gist of the embodiment unnecessarily vague.

Furthermore, in describing components of an embodiments, terms, such as a first, a second, A, B, (a), and (b), may be used. Such terms are used only to distinguish one component from the other component, and the essence, order, or sequence of a corresponding component is not limited by the terms. When it is said that one component is “connected”, “combined”, or “coupled” to the other component, the one component may be directly connected or coupled to the other component, but it should also be understood that a third component may be “connected”, “combined”, or “coupled” between the two components.

A component included in any one embodiment and a component including a common function are described using the same name in another embodiment. Unless described otherwise, a description written in any one embodiment may be applied to another embodiment, and a detailed description in a redundant range is omitted.

A system for predicting a health risk according to embodiments of the present disclosure may be implemented by at least one computer device. In this case, a computer program according to an embodiment of the present disclosure may be installed and driven in the computer device. The computer device may perform a method of predicting a health risk according to embodiments of the present disclosure under the control of the driven computer program. The aforementioned computer program may be stored in a computer-readable recording medium in order to execute the method of predicting a health risk by being coupled to the computer device.

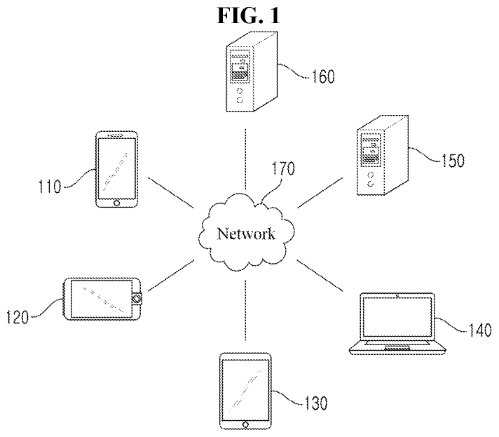

FIG. 1 is a diagram illustrating an example of a network environment according to an embodiment of the present disclosure. The network environment of FIG. 1 illustrates an example including a plurality of electronic devices 110, 120, 130, and 140, a plurality of servers 150 and 160, and a network 170. FIG. 1 is an example for describing the present disclosure, and the number of electronic devices or the number of servers is not limited to that of FIG. 1. Furthermore, the network environment of FIG. 1 merely describes one of environments applicable to the present embodiments, and an environment applicable to the present embodiments is not limited to the network environment of FIG. 1.

Each of the plurality of electronic devices 110, 120, 130 and 140 may be a stationary terminal or a mobile terminal implemented as a computer device. For example, the plurality of electronic devices 110, 120, 130 and 140 may include a smartphone, a mobile phone, a navigation device, a computer, a laptop computer, a device for digital broadcasting, personal digital assistants (PDA), a portable multimedia player (PMP), a tablet PC, etc. For example, in FIG. 1, a shape of a smartphone is illustrated as being an example of the electronic device 110. However, in embodiments of the present disclosure, the electronic device 110 may mean one of various physical computer devices capable of communicating with other electronic devices 120, 130 and 140 and/or the servers 150 and 160 over the network 170 substantially using a wireless or wired communication method.

The communication method is not limited, and may include short-distance wireless communication between devices in addition to communication methods using communication networks (e.g., a mobile communication network, wired Internet, wireless Internet, and a broadcasting network) which may be included in the network 170. For example, the network 170 may include one or more given networks of a personal area network (PAN), a local area network (LAN), a campus area network (CAN), a metropolitan area network (MAN), a wide area network (WAN), a broadband network (BBN), and the Internet. Furthermore, the network 170 may include one or more of network topologies, including a bus network, a star network, a ring network, a mesh network, a star-bus network, and a tree or hierarchical network, but is not limited thereto.

Each of the servers 150 and 160 may be implemented as a computer device or a plurality of computer devices, which provides an instruction, a code, a file, content, or a service through communication with the plurality of electronic devices 110, 120, 130 and 140 over the network 170. For example, the server 150 may be a system that provides the plurality of electronic devices 110, 120, 130, and 140 with services (e.g., a health management service, an instant messaging service, a financial service, a game service, a group call service (or voice conference service), a messaging service, a mailing service, a social network service, a map service, a translation service, a payment service, a search service, and a content provision service).

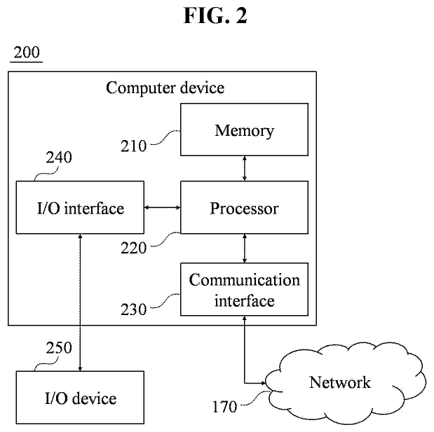

FIG. 2 is a block diagram illustrating an example of a computer device according to an embodiment of the present disclosure. Each of the plurality of electronic devices 110, 120, 130 and 140 or each of the servers 150 and 160 may be implemented as a computer device 200 illustrated in FIG. 2.

As illustrated in FIG. 2, the computer device 200 may include a memory 210, a processor 220, a communication interface 230 and an input/output (I/O) interface 240. The memory 210 is a computer-readable medium, and may include permanent mass storage devices, such as a random access memory (RAM), a read only memory (ROM) and a disk drive. In this case, the permanent mass storage device, such as a ROM and a disk drive, may be included in the computer device 200 as a permanent storage device separated from the memory 210. Furthermore, an operating system and at least one program code may be stored in the memory 210. Such software components may be loaded from a computer-readable medium, separated from the memory 210, to the memory 210. Such a separate computer-readable medium may include computer-readable recording media, such as a floppy drive, a disk, a tape, a DVD/CD-ROM drive, and a memory card. In another embodiment, software components may be loaded onto the memory 210 through the communication interface 230 not a computer-readable medium. For example, the software components may be loaded onto the memory 210 of the computer device 200 based on a computer program installed by files received over the network 170.

The processor 220 may be configured to process instructions of a computer program by performing basic arithmetic, logic and input/output (I/O) operations. The instructions may be provided to the processor 220 by the memory 210 or the communication interface 230. For example, the processor 220 may be configured to execute received instructions based on a program code stored in a recording device, such as the memory 210.

The communication interface 230 may provide a function for enabling the computer device 200 to communicate with other devices (e.g., the aforementioned storage devices) over the network 170. For example, a request, a command, data or a file generated by the processor 220 of the computer device 200 based on a program code stored in a recording device, such as the memory 210, may be provided to other devices over the network 170 under the control of the communication interface 230. Inversely, a signal, a command, data or a file from another device may be received by the computer device 200 through the communication interface 230 of the computer device 200 over the network 170. A signal, a command or a file received through the communication interface 230 may be transmitted to the processor 220 or the memory 210. A file received through the communication interface 230 may be stored in a storage device (e.g., the aforementioned permanent storage device) which may be further included in the computer device 200.

The I/O interface 240 may be means for an interface with an I/O device 250. For example, the input device may include a device, such as a microphone, a keyboard, or a mouse. The output device may include a device, such as a display or a speaker. For another example, the I/O interface 240 may be means for an interface with a device in which functions for input and output have been integrated into one, such as a touch screen. At least one of the I/O devices 250, together with the computer device 200, may be configured as a single device. For example, the I/0 device may be implemented in a form in which a touch screen, a microphone, a speaker, etc. are included in the computer device 200 like a smartphone.

Furthermore, in other embodiments, the computer device 200 may include components greater or smaller than the components of FIG. 2. However, it is not necessary to clearly illustrate most of conventional components. For example, the computer device 200 may be implemented to include at least some of the I/O devices 250 or may further include other components, such as a transceiver and a database.

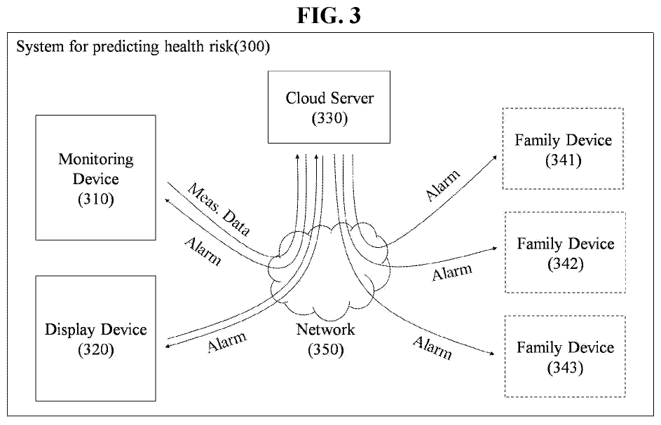

FIG. 3 is a diagram illustrating an example of a system 300 for predicting a health risk according to an embodiment of the present disclosure. The system 300 for predicting a health risk according to the present disclosure is a system for helping a user to secure the time to handle a health risk and to avoid a risk situation by previously predicting the user's health risk. As illustrated in the embodiment of FIG. 3, the system 300 may include a monitoring device 310, a display device 320, a cloud server 330 and a plurality of family devices 341 to 343. FIG. 3 illustrates three family devices like the plurality of family devices 341 to 343, but the number of family devices is not limited to three.

The monitoring device 310 may collect one or more health condition indices (HCIs) and transmit the HCIs to the cloud server 330. In this case, the HCI may include values of blood pressure, oxygen saturation, blood glucose, a heat rate, a body temperature, etc. measured with respect to an object through a bio sensor or digitized values on which the values may be estimated. In this case, the object may basically mean a human body, but the present disclosure is not limited thereto. For example, an animal, such as livestock, may be included in the object.

The monitoring device 310 includes the bio sensor, and may directly measure an HCI from an object or may receive an HCI of an object measured by an external device. The external device may be an insertion type sensor inserted into the body of an object, for example, but the present disclosure is not limited thereto. For example, the external device may be an external sensor for measuring an HCI from an object outside the body of the object and transmitting the HCI. The monitoring device 310 may transmit, to the cloud server 330, an HCI directly measured or received from an external device as described above over a network 350. In this case, the network 350 may correspond to the network 170 described with reference to FIGS. 1 and 2.

The network 350 consists of one or more communication channels. Each of the communication channels may be a wired or wireless communication channel. The communication channel may correspond to WiFi, Ethernet, a mobile network, a public switched telephone network (PSTN), etc., but the present disclosure is not limited thereto.

The cloud server 330 may generate time-series data by accumulating received HCIs. The time-series data may be represented in the form of a two-dimensional array consisting of HCIs within a given time interval.

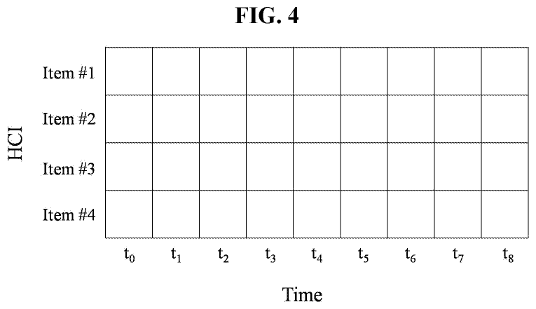

FIG. 4 is a diagram illustrating an example of time-series data according to an embodiment of the present disclosure. FIG. 4 illustrates an example in which a plurality of items of an HCI is represented in the form of a two-dimensional array over time.

Referring back to FIG. 3, the cloud server 330 may predict an HCI after several minutes to several months by analyzing generated time-series data by using an artificial intelligence algorithm.

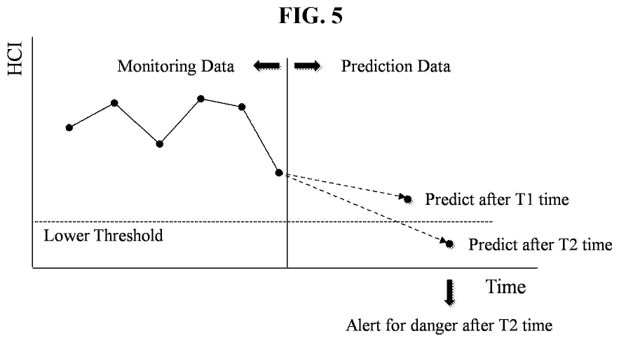

FIG. 5 is a diagram illustrating an example in which HCIs are predicted according to an embodiment of the present disclosure. The embodiment of FIG. 5 illustrates HCIs after a time T1 and a time T2, which were predicted using data monitored by the cloud server 330 (e.g., time-series data generated by accumulating HCIs received from the monitoring device 310). In this case, since a prediction value after the time T2 is equal to or smaller than a lower threshold, the cloud server 330 may generate a danger alert signal after the time T2, and may transmit the generated danger alert signal to the monitoring device 310, the display device 320 and at least one of the plurality of family devices 341 to 343.

The display device 320 and the plurality of family devices 341 to 343 may notify a user of the risk situation by generating a sound, vibration, light, etc. based on the received danger alert signal. The display device 320 may be a smartphone, a wearable device, etc. The family device (i.e., at least one of 341 to 343) may be a smartphone, a wearable device, a PC, a terminal device for a hospital, etc. However, a device for notifying a user of a risk situation based on a danger alert signal is not limited to the display device 320 or the plurality of family devices 341 to 343. A method for providing notification of a risk situation is also not limited to a sound, vibration, light, etc.

The AI algorithm of the cloud server 330 that analyzes time-series data may include one or more of various algorithms, such as Multi-layer Perceptron (MLP), a deep neural network (DNN), a convolutional neural network (CNN), a recurrent neural network (RNN), a group convolutional neural network (G-CNN) and a recurrent convolutional neural network (R-CNN), and is not limited to a specific algorithm.

For example, the cloud server 330 may generate an HCI prediction model by training an AI algorithm model through machine learning using learning data. Supervised learning or unsupervised learning may be used as the machine learning, and reinforcement learning may be used during the unsupervised learning, but this is merely an example. A learning method of the present disclosure is not limited thereto.

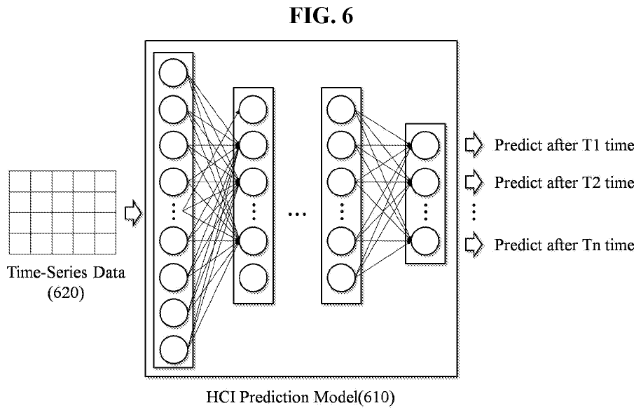

FIG. 6 is a concept view in which HCIs are expected through an HCI prediction model 610 according to an embodiment of the present disclosure. The HCI prediction model 610 may output prediction value after respective times through a calculation process within the HCI prediction model 610 when time-series data 620 is received. Future times T1, T2, . . . , Tn when HCIs will be predicted may be preset in a model selection process. Accordingly, learning data may be prepared. According to circumstances, a model for predicting an HCH in only one time T1 may be produced. As in the embodiment of FIG. 6, a model for predicting an HCI in several times may be produced.

In the aforementioned embodiments, an example in which the cloud server 330 generates time-series data and processes prediction has been described. However, in some embodiments, the generation of time-series data and the prediction may be processed by the monitoring device 310.

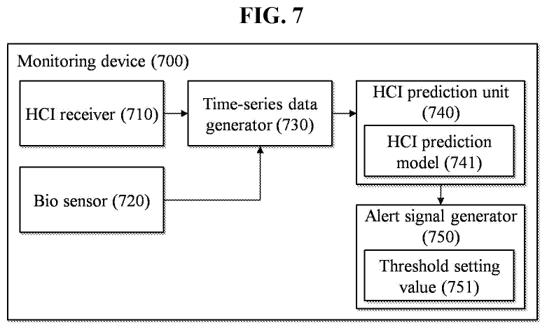

FIG. 7 is a diagram illustrating an example of internal components of a monitoring device 700 according to an embodiment of the present disclosure. The monitoring device 700 according to the present disclosure may include an HCI receiver 710, a bio sensor 720, a time-series data generator 730, an HCI prediction unit 740 and an alert signal generator 750. The embodiment of FIG. 7 describes a case where the monitoring device 700 includes both the HCI receiver 710 and the bio sensor 720. However, in some embodiments, the monitoring device 700 may include only one of the HCI receiver 710 and the bio sensor 720.

The HCI receiver 710 may receive one or more HCIs for an object from an external device. In order to generate time-series data, the HCI receiver 710 may receive an HCI at a given time interval.

The bio sensor 720 may measure one or more HCIs for an object. Even in this case, in order to generate time-series data, the bio sensor 720 may measure an HCI at a given time interval.

The measurement of an HCI in the bio sensor 720 or the external device may be performed using at least one of already well-known methods. For example, the bio sensor 720 or the external device may measure, as one type of HCI, a concentration of analytes based on a change in the relative permittivity of a biological tissue within a living body.

The time-series data generator 730 may receive an HCI from the HCI receiver 710 and/or the bio sensor 720, and may generate time-series data. For example, the time-series data generator 730 may generate time-series data by accumulating an HCI at given time intervals in the form of a two-dimensional array.

The HCI prediction unit 740 may calculate an HCI prediction value in a future time based on time-series data generated by the time-series data generator 730, by using an HCI prediction model 741.

The alert signal generator 750 may compare an HCI prediction value, calculated by the HCI prediction unit 740, with a preset threshold setting value 751, and may generate a danger alert signal when the HCI prediction value is out of the threshold setting value 751. In FIG. 5, only a lower threshold has been described, but an upper threshold may be present or both a lower threshold and an upper threshold may be present depending on the type of HCI.

In some embodiments, the monitoring device 700 may further include one or more of an alert signal output unit (not illustrated), a display (not illustrated) and a communication unit (not illustrated). For example, the monitoring device 700 may output, through the alert signal output unit, a danger alert signal generated by the alert signal generator 750. In another embodiment, the monitoring device 700 may output a danger alert signal through a display or may transmit a danger alert signal to the display device 320 or the plurality of family devices 341 to 343 described with reference to FIG. 3 through the communication unit. In this case, the display device 320 or the plurality of family devices 341 to 343 may output the received danger alert signal instead of the monitoring device 700.

As described above, the alert signal output unit may output a danger alert signal generated by the alert signal generator 750. The danger alert signal may be output in the form of a sound, vibration, light, etc., but the present disclosure is not limited thereto.

The display may display at least one of an HCI, an HCI prediction value and a danger alert signal.

The communication unit may transmit at least one of an HCI, an HCI prediction value and a danger alert signal to another device (e.g., the display device 320, the cloud server 330 and at least one of the plurality of family devices 341 to 343).

Furthermore, in some embodiments, the generation of time-series data and the prediction may be processed by the display device 320.

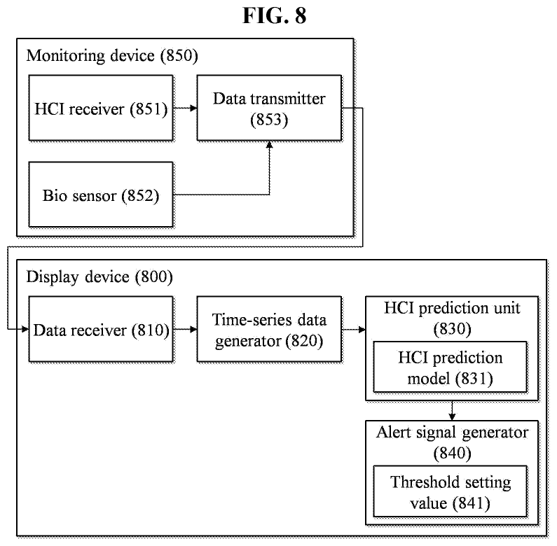

FIG. 8 is a diagram illustrating an example of internal components of a display device 800 according to an embodiment of the present disclosure. The display device 800 according to the present disclosure may include a data receiver 810, a time-series data generator 820, an HCI prediction unit 830 and an alert signal generator 840. In the present embodiment, a monitoring device 850 may include an HCI receiver 851, a bio sensor 852 and a data transmitter 853. In this case, the HCI receiver 851 and the bio sensor 852 may correspond to the HCI receiver 710 and the bio sensor 720 described with reference to FIG. 7, respectively. The data transmitter 853 may be implemented to transmit, to the display device 800, an HCI collected by the HCI receiver 851 and/or the bio sensor 852.

In this case, the data receiver 810 may receive an HCI transmitted by the monitoring device 850 through the data transmitter 853. In this case, the time-series data generator 820, the HCI prediction unit 830 and the alert signal generator 840 may correspond to the time-series data generator 730, the HCI prediction unit 740 and the alert signal generator 750 described with reference to FIG. 7, respectively.

In other words, the time-series data generator 820 may generate time-series data by using an HCI received by the data receiver 810. The HCI prediction unit 830 may calculate an HCI prediction value in a future time by inputting the time-series data to the HCI prediction model 831. Furthermore, the alert signal generator 840 may compare the HCI prediction value, calculated by the HCI prediction unit 830, with a preset threshold setting value 841, and may generate a danger alert signal when the HCI prediction value is out of the threshold setting value 841.

In this case, the display device 800 may further include an alert signal output unit (not illustrated), a display (not illustrated) and a communication unit (not illustrated). The alert signal output unit may output a danger alert signal generated by the alert signal generator 840. The display may display at least one of an HCI, an HCI prediction value, and a danger alert signal. Furthermore, the communication unit may transmit at least one of an HCI, an HCI prediction value, and a danger alert signal to another device (e.g., the cloud server 330 and at least one of the plurality of family devices 341 to 343).

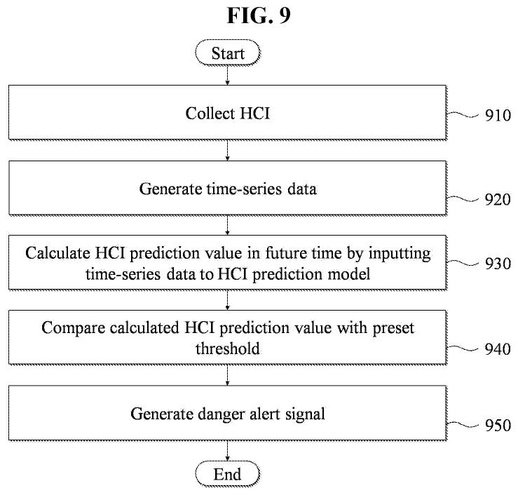

FIG. 9 is a flowchart illustrating an example of a method of predicting a health risk according to an embodiment of the present disclosure. The method of predicting a health risk according to the present disclosure may be performed by the computer device 200. In this case, the processor 220 of the computer device 200 may be implemented to execute a control instruction based on a code of an operating system or a code of at least one computer program included in the memory 210. In this case, the processor 220 may control the computer device 200 so that the computer device 200 performs steps 910 to 950 included in the method of FIG. 9 in response to a control instruction provided by a code stored in the computer device 100. In this case, the computer device 200 may correspond to the cloud server 330 of FIG. 1, the monitoring device 700 of FIG. 7 or the display device 800 of FIG. 8.

In step 910, the computer device 200 may collect an HCI. In this case, to collect an HCI may include receiving the HCI from an external device and/or measuring the HCI through the bio sensor. For example, if the computer device 200 corresponds to the cloud server 330 of FIG. 1 or the display device 800 of FIG. 8, to collect an HCI may correspond to receiving the HCI from the monitoring device 310 or 850. In contrast, if the computer device 200 corresponds to the monitoring device 700 of FIG. 7, to collect an HCI may correspond to receiving the HCI from an external sensor and/or measuring the HCI through the bio sensor 720 of the monitoring device 700.

In step 920, the computer device 200 may generate time-series data. As described above, the computer device 200 may generate time-series data by accumulating an HCI at given time intervals in the form of a two-dimensional array. If HCIs include a plurality of types, the computer device 200 may generate time-series data by accumulating the HCIs at given time intervals for each type.

In step 930, the computer device 200 may calculate an HCI prediction value in a future time by inputting the time-series data to an HCI prediction model. As described above, the HCI prediction model may be generated to learn an AI algorithm model through machine learning using learning data, receive time-series data and output an HCI prediction value in one or more future times.

In step 940, the computer device 200 may compare the calculated HCI prediction value with a preset threshold. In this case, when the calculated HCI prediction value is out of the preset threshold, step 950 may be performed. As described above, the threshold may include a case where an upper threshold, a case where a lower threshold is present, and a case where both a lower threshold and an upper threshold are present depending on the type of HCI. In some embodiments, the threshold may be present in the form of a range between a first threshold and a second threshold. In this case, when an HCI prediction value is a value between the first threshold and the second threshold, the HCI prediction value may be determined to be out of the threshold.

In step 950, the computer device 200 may generate a danger alert signal. For example, if the calculated HCI prediction value is determined to be out of the preset threshold in step 940, in step 950, the computer device 200 may generate a danger alert signal. If the calculated HCI prediction value is determined to be not out of the preset threshold, step 910 may be repeatedly performed or the process may be terminated.

Furthermore, in some embodiments, the system 300 for predicting a health risk may generate a guide for improving a lifestyle for the purpose of continuous health management and provide a user with the guide, in addition to previously predicting and providing notification of a health risk.

For example, referring back to FIG. 3, the cloud server 330 may generate time-series data by receiving an HCI from the monitoring device 310 and accumulating the HCI. As described above, the time-series data may be represented in the form of a two-dimensional array consisting of HCIs within a given time interval.

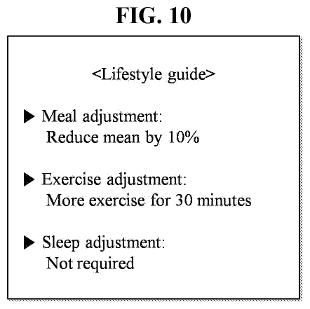

In this case, the cloud server 330 may generate and provide a lifestyle guide by inputting the time-series data to a lifestyle guide model. The lifestyle guide may consist of one or more of items, such as a meal adjustment guide, an exercise adjustment guide, and a sleep adjustment guide, and may include a change recommendation value of each item. FIG. 10 is a diagram illustrating an example of lifestyle guides according to an embodiment of the present disclosure. The lifestyle guide of FIG. 10 includes a change announcement value of 10% for a corresponding item as a guide for meal adjustment, and includes a change announcement value (more 30 minutes per day) for a corresponding item as a guide for exercise adjustment. Furthermore, the lifestyle guide of FIG. 10 further includes a guide for sleep adjustment. In this case, FIG. 10 illustrates that sleep adjustment is not required.

The cloud server 330 may transmit the lifestyle guide to the monitoring device 310, the display device 320 and at least one of the plurality of family devices 341 to 343.

In this case, the display device 320 and the plurality of family devices 341 to 343 may display the received lifestyle guide on a screen, or may notify a user of the received lifestyle guide in the form of a sound, vibration, light, etc. As described above, the display device 320 may be a smartphone, a wearable device, etc. Each of the plurality of family devices 341 to 343 may be a smartphone, a wearable device, a PC, a terminal device for a hospital, etc., but the present disclosure is not limited thereto.

Various models, such as linear regression, Multi-layer Perceptron (MLP), a deep neural network (DNN), a convolutional neural network (CNN), a recurrent neural network (RNN), a group convolutional neural network (G-CNN), a recurrent convolutional neural network (R-CNN), a Bayesian neural network (BNN), may be applied to the lifestyle guide model for analyzing time-series data in the cloud server 330, but the present disclosure is not limited to a specific model.

Furthermore, the cloud server 330 may construct an AI model through machine learning using learning data. Supervised learning or unsupervised learning may be used as the machine learning, and reinforcement learning may be used during the unsupervised learning, but a learning method of the present disclosure is not limited thereto.

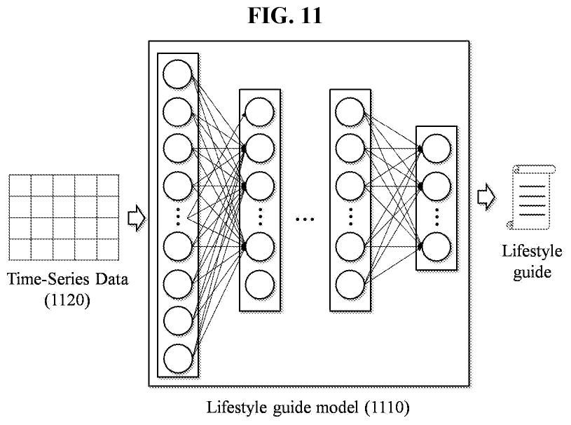

FIG. 11 is a diagram illustrating an example of a lifestyle guide model 1110 according to an embodiment of the present disclosure. The lifestyle guide model 1110 may output a lifestyle guide through a calculation process within the lifestyle guide model 1110 when receiving time-series data 1120. Data obtained by previously accumulating an HCI for a given time and a pair of answers of a corresponding lifestyle guide may be previously generated as learning data. The lifestyle guide model 1110 may previously learn such learning data.

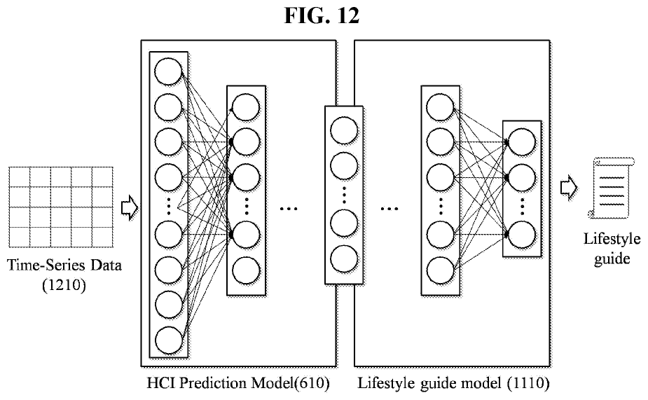

FIG. 12 is a diagram illustrating another example of a lifestyle guide model according to an embodiment of the present disclosure. The embodiment of FIG. 12 illustrates an example in which different AI models of the HCI prediction model 610 and the lifestyle guide model 1110 are sequentially connected. Time-series data 1210 may be input to the HCI prediction model 610. An output value of the HCI prediction model 610 may be input to the lifestyle guide model 1110 again. Thereafter, the lifestyle guide model 1110 may generate a lifestyle guide as an output value.

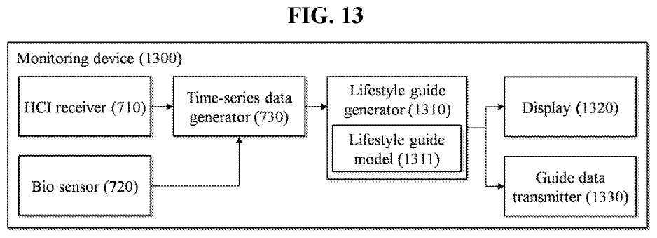

FIG. 13 is a diagram illustrating another example of internal components of a monitoring device 1300 according to an embodiment of the present disclosure. The monitoring device 1300 according to the present disclosure may include the HCI receiver 710, the bio sensor 720, the time-series data generator 730, a lifestyle guide generator 1310, a display 1320 and a guide data transmitter 1330. In this case, the HCI receiver 710, the bio sensor 720, and the time-series data generator 730 may be the same components as the HCI receiver 710, the bio sensor 720 and the time-series data generator 730 described in the embodiment of FIG. 7, respectively. In some embodiments, the monitoring device 1300 may be implemented in a form to include all the components (e.g., the HCI receiver 710, the bio sensor 720, the time-series data generator 730, the HCI prediction unit 740 and the alert signal generator 750) of the monitoring device 700 of FIG. 7 and to further include the lifestyle guide generator 1310, the display 1320 and the guide data transmitter 1330. However, in the embodiment of FIG. 13, an example in which the monitoring device 1300 includes the lifestyle guide generator 1310, the display 1320 and the guide data transmitter 1330 instead of the HCI prediction unit 740 and the alert signal generator 750 is described.

As described above, the HCI receiver 710 may receive one or more HCIs for an object from an external device. In order to generate time-series data, the HCI receiver 710 may receive an HCI at a given time interval.

The bio sensor 720 may measure one or more HCIs for an object. Even in this case, in order to generate time-series data, the bio sensor 720 may measure an HCI at a given time interval.

The measurement of the HCI in the bio sensor 720 or the external device may be performed using at least one of well-known measurement methods. For example, the bio sensor 720 or the external device may measure, as one type of HCI, a concentration of analytes based on a change in relative permittivity of a biological tissue within a living body.

In this case, the embodiment of FIG. 13 describes a case where the monitoring device 1300 includes both the HCI receiver 710 and the bio sensor 720. However, in some embodiments, the monitoring device 1300 may include only one of the HCI receiver 710 and the bio sensor 720.

The time-series data generator 730 may receive an HCI from the HCI receiver 710 and/or the bio sensor 720 and generate time-series data. For example, the time-series data generator 730 may generate time-series data by accumulating an HCI at given time intervals in the form of a two-dimensional array.

The lifestyle guide generator 1310 may generate a lifestyle guide based on the time-series data generated by the time-series data generator 730, by using a lifestyle guide model 1311.

The display 1320 may display the generated lifestyle guide.

The guide data transmitter 1330 may transmit the generated lifestyle guide to an external device, such as the display device 320 or the cloud server 330.

In some embodiments, the monitoring device 1300 may be implemented to include only one of the display 1320 and the guide data transmitter 1330.

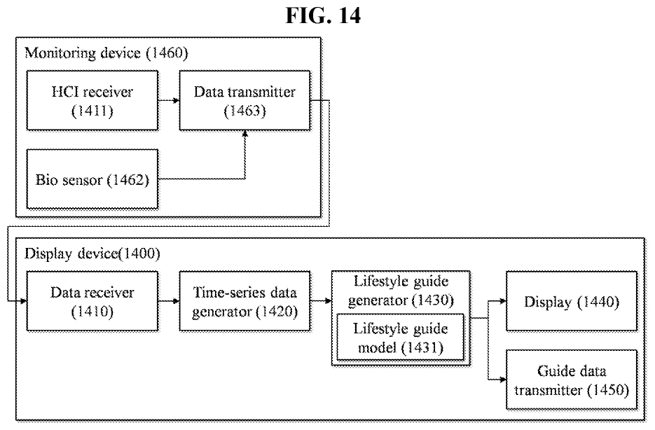

FIG. 14 is a diagram illustrating another example of internal components of a display device 1400 according to an embodiment of the present disclosure. The display device 1400 according to the present disclosure may include a data receiver 1410, a time-series data generator 1420, a lifestyle guide generator 1430, a display 1440 and a guide data transmitter 1450. In the present embodiment, a monitoring device 1460 may include an HCI receiver 1461, a bio sensor 1462 and a data transmitter 1463. In this case, the HCI receiver 1461 and the bio sensor 1462 may correspond to the HCI receiver 710 and the bio sensor 720 described with reference to FIG. 13, respectively. The data transmitter 1463 may be implemented to transmit, to the display device 1400, an HCI collected by the HCI receiver 1461 and/or the bio sensor 1462.

In this case, the data receiver 1410 may receive the HCI transmitted by the monitoring device 1460 through the data transmitter 1463. In this case, the time-series data generator 1420, the lifestyle guide generator 1430, the display 1440 and the guide data transmitter 1450 may correspond to the time-series data generator 730, the lifestyle guide generator 1310, the display 1320 and the guide data transmitter 1330 described with reference to FIG. 13, respectively.

In other words, the time-series data generator 1420 may generate time-series data based on the HCI received by the data receiver 1410. The lifestyle guide generator 1430 may generate a lifestyle guide based on the time-series data generated by the time-series data generator 1420 by using a lifestyle guide model 1431. Furthermore, the display 1440 may display the generated lifestyle guide. The guide data transmitter 1450 may transmit the generated lifestyle guide to an external device, such as the display device 320 or the cloud server 330. In some embodiments, the display device 1400 may be implemented to include only any one of the display 1440 and the guide data transmitter 1450.

As described above, according to the embodiments of the present disclosure, when a risk, such as a hypoglycemia shock, dysarteriotony, reduced oxygen saturation, a sudden change in the heart rate, or an abnormal body temperature, is predicted based on a change in a future health state, a user is previously warned of the risk so that the user can avoid the risk by securing the time to handle the risk.

The aforementioned system or device may be implemented as a hardware component, a software component and/or a combination of a hardware component and a software component. For example, the device and components described in the embodiments may be implemented using one or more general-purpose computers or special-purpose computers, for example, a processor, a controller, an arithmetic logic unit (ALU), a digital signal processor, a microcomputer, a field programmable gate array (FPGA), a programmable logic unit (PLU), a microprocessor or any other device capable of executing or responding to an instruction. A processing device may perform an operating system (OS) and one or more software applications executed on the OS. Furthermore, the processing device may access, store, manipulate, process and generate data in response to the execution of software. For convenience of understanding, one processing device has been illustrated as being used, but a person having ordinary knowledge in the art may understand that the processing device may include a plurality of processing components and/or a plurality of types of processing components. For example, the processing device may include a plurality of processors or one processor and one controller. Furthermore, other processing configurations, such as a parallel processor, are also possible.

Software may include a computer program, a code, an instruction or a combination of one or more of them, and may configure a processor so that it operates as desired or may instruct processors independently or collectively. Software and/or data may be embodied in any type of a machine, component, physical device, virtual equipment, or computer storage medium or device so as to be interpreted by the processor or to provide an instruction or data to the processor. The software may be distributed to computer systems connected over a network and may be stored or executed in a distributed manner. The software and data may be stored in one or more computer-readable recording media.

The method according to the embodiment may be implemented in the form of a program instruction executable by various computer means and stored in a computer-readable recording medium. The computer-readable recording medium may include a program instruction, a data file and a data structure alone or in combination. The program instructions stored in the medium may be specially designed and constructed for the present disclosure, or may be known and available to those skilled in the field of computer software. Examples of the computer-readable storage medium include magnetic media such as a hard disk, a floppy disk and a magnetic tape, optical media such as a CD-ROM and a DVD, magneto-optical media such as a floptical disk, and hardware devices specially configured to store and execute program instructions such as a ROM, a RAM, and a flash memory. Examples of the program instructions include not only machine language code that is constructed by a compiler but also high-level language code that can be executed by a computer using an interpreter or the like.

As described above, although the embodiments have been described in connection with the limited embodiments and the drawings, those skilled in the art may modify and change the embodiments in various ways from the description. For example, proper results may be achieved although the aforementioned descriptions are performed in order different from that of the described method and/or the aforementioned components, such as the system, configuration, device, and circuit, are coupled or combined in a form different from that of the described method or replaced or substituted with other components or equivalents.

Accordingly, other implementations, other embodiments, and the equivalents of the claims fall within the scope of the claims.

1. A method of predicting, by a computer device comprising at least one processor, a health risk, the method comprising:
collecting, by the at least one processor, a health condition index;
generating, by the at least one processor, time-series data by accumulating the health condition index at given time intervals;
calculating, by the at least one processor, a health condition index prediction value in a future time by inputting the generated time-series data to a health condition index prediction model;
comparing, by the at least one processor, the calculated health condition index prediction value with a preset threshold;
generating, by the at least one processor, a danger alert signal when the calculated health condition index prediction value is out of the threshold; and
generating a lifestyle guide by inputting the generated time-series data to a lifestyle guide model by:
training the lifestyle guide with learning data, wherein the learning data is an accumulated health condition index for a given time and a pair of answers of a corresponding lifestyle guide;
constructing an AI model through machine learning with a cloud server using the learning data; and
outputting the lifestyle guide.

2. The method of claim 1, wherein collecting the health condition index comprises receiving the health condition index of an object from an external device or measuring the health condition index of the object through a bio sensor.

3. The method of claim 1, wherein generating the time-series data comprises generating the time-series data by accumulating the health condition index at given time intervals in the form of a two-dimensional array for each type.

4. The method of claim 1, wherein the health condition index prediction model is trained to receive the time-series data obtained by accumulating the health condition index over time and to output a prediction value for a health condition index in at least one future time after the time-series data.

5. The method of claim 1, wherein comparing the calculated health condition index prediction value with the preset threshold comprises determining that the calculated health condition index prediction value is out of the preset threshold, when:
the calculated health condition index prediction value is smaller than a preset lower threshold,
the calculated health condition index prediction value is greater than a preset upper threshold, or
the calculated health condition index prediction value is included in a preset threshold range.

6. The method of claim 1, further comprising outputting, by the at least one processor, the generated danger alert signal.

7. The method of claim 1, further comprising displaying, by the at least one processor, at least one of the collected health condition index, the calculated health condition index prediction value and the danger alert signal.

8. The method of claim 1, further comprising transmitting, by the at least one processor, at least one of the collected health condition index, the calculated health condition index prediction value and the danger alert signal to an external device.

9. (canceled)

10. The method of claim 1, wherein generating the lifestyle guide further comprises:
inputting the generated time-series data to the health condition index prediction model,
inputting an output of the health condition index prediction model to the lifestyle guide model again, and
generating an output value of the lifestyle guide model as the lifestyle guide.

11. A computer device comprising:
at least one processor implemented to execute computer-readable instructions, the at least one processor is implemented to:
collect a health condition index,
generate time-series data by accumulating the health condition index at given time intervals,
calculate a health condition index prediction value in a future time by inputting the generated time-series data to a health condition index prediction model,
compare the calculated health condition index prediction value with a preset threshold,
generate a danger alert signal when the calculated health condition index prediction value is out of the threshold; and
generate a lifestyle guide by inputting the generated time-series data to a lifestyle guide model by:
training the lifestyle guide with learning data, wherein the learning data is an accumulated health condition index for a given time and a pair of answers of a corresponding lifestyle guide;
constructing an AI model through machine learning with a cloud server using the learning data; and
outputting the lifestyle guide.

12. The computer device of claim 11, wherein in order to collect the health condition index, the at least one processor receives the health condition index of an object from an external device or measures the health condition index of the object through a bio sensor.

13. The computer device of claim 11, wherein in order to generate the time-series data, the at least one processor generates the time-series data by accumulating the health condition index at given time intervals in the form of a two-dimensional array for each type.

14. The computer device of claim 11, wherein the health condition index prediction model is trained to receive the time-series data obtained by accumulating the health condition index over time and to output a prediction value for a health condition index in at least one future time after the time-series data.

15. The computer device of claim 11, wherein in order to compare the calculated health condition index prediction value with the preset threshold, the at least one processor determines that the calculated health condition index prediction value is out of the preset threshold, when:
the calculated health condition index prediction value is smaller than a preset lower threshold,
the calculated health condition index prediction value is greater than a preset upper threshold, or
the calculated health condition index prediction value is included in a preset threshold range.

16. (canceled)

17. The computer device of claim 1, wherein in order to generate the lifestyle guide, the at least one processor
inputs the generated time-series data to the health condition index prediction model,
inputs an output of the health condition index prediction model to the lifestyle guide model again, and
generates an output value of the lifestyle guide model as the lifestyle guide.
